(19) United States
(12) Patent Application Publication
(10) Pub. No.: US 2020/0370022 A1
(43) Pub. Date: Nov. 26, 2020

(54) MALIGNANT HEMATOPOIETIC CELL MICROCOMPARTMENT AND METHOD FOR PREPARING SUCH A MICROCOMPARTMENT

(71) Applicants: UNIVERSITE DE BORDEAUX, BORDEAUX (FR); CENTRE NATIONAL DE LA RECHERCHE SCIENTIFIQUE, PARIS (FR); INSERM (INSTITUT NATIONAL DE LA SANTÉ ET DE LA RECHERCHE MÉDICALE), PARIS (FR); INSTITUT D'OPTIQUE THEORIQUE ET APPLIQUEE, PALAISEAU CEDEX (FR); INSTITUT BERGONIE, BORDEAUX (FR); UNIVERSITE DE RENNES 1, RENNES CEDEX (FR); ETABLISSEMENT FRANCAIS DU SANG, LA PLAINE SAINT DENIS CEDEX (FR); CENTRE HOSPITALIER UNIVERSITAIRE (CHU) DE RENNES, RENNES CEDEX 9 (FR)

(72) Inventors: LAURENCE BRESSON-BEPOLDIN, SAINT MEDARD EN JALLES (FR); SIMON LATOUR, BORDEAUX (FR); ISABELLE MAHOUCHE, LES BILLAUX (FR); PIERRE NASSOY, BORDEAUX (FR); KEVIN ALESSANDRI, BORDEAUX (FR); KARIN TARTE, RENNES (FR); FRÉDÉRIC MOURCIN, SAINT-MALO (FR)

(21) Appl. No.: 16/603,264

(22) PCT Filed: Apr. 5, 2018
(86) PCT No.: PCT/FR2018/050855

§ 371 Date: Oct. 7, 2019

(30) Foreign Application Priority Data
Apr. 7, 2017 (FR) 1753067

(51) Int. Cl. C12N 5/09; C12N 5/00; C12N 5/0781; G01N 33/50

(52) U.S. Cl. CPC C12N 5/0694; C12N 5/0012; C12N 5/0062; C12N 5/0068; C12N 5/0635; C12N 2533/90; C12N 2502/1358; C12N 2503/02; C12N 2503/04; C12N 2533/74; G01N 33/5011

(57) ABSTRACT

The invention relates to a process for preparing cellular microcompartments comprising a hydrogel capsule surrounding a cluster of lymphomatous cells. The invention also relates to such a cellular microcompartment and the use thereof for screening anti-cancer molecules.

The invention relates to a process for preparing cellular microcompartments comprising malignant haematopoietic cells. The invention also relates to such cellular microcompartments and the use thereof, particularly in the pharmaceutical field, for screening and identifying molecules of interest likely to treat malignant haemopathy.

Cancers of haematopoietic tissues, or malignant haemopathies, are characterized by a disorder of the multiplication and differentiation of cells of a blood line. Among the most common malignant haemopathies are leukaemias and lymphomas.

Leukaemia is a cancer of bone marrow cells, characterized by an abnormal and massive proliferation of white blood cell precursors that are not completely differentiated, to the detriment of red blood cells, normal white blood cells and platelets. There are four main types of leukaemia: acute lymphoblastic leukaemia (ALL), chronic lymphoid leukaemia (CLL), acute myeloblastic leukaemia (AML) and chronic myeloid leukaemia (CIVIL).

Lymphoma is a group of cancers of the lymphatic system that originates in a secondary lymphoid organ and can spread to all parts of the lymphatic system. There are two main types of lymphomas: Hodgkin's lymphoma and non-Hodgkin's lymphomas (NHL). NHL are cancers whose incidence has been increasing for 40 years in developed countries and which rank 10^{th} among cancers in terms of frequency.

The currently available treatments, which most often combine chemotherapy and immunotherapy, are only effective in a proportion of patients, due to the existence of many resistances or relapses. One reason for this is the lack of relevant cellular models to test candidate molecules. Indeed, at present, candidate molecules are tested in vitro on cell lines in suspension (for non-adherent cells) or in monolayer (for adherent cells). These two-dimensional (2D) cellular models are not representative of lymphomas since, unlike cells within the tumour, all cells have identical access to nutrients and oxygen as well as to candidate molecules. In addition, lymphomas form and evolve within secondary lymphoid organs comprising several types of cells (microenvironmental cells and lymphomatous cells), which interact with each other in an extracellular matrix via soluble and membrane molecules and are subjected to biomechanical forces. All these elements have an impact on the development of lymphomas, but also on the response to treatment. However, 2D models do not allow these phenomena to be reproduced and are therefore only weakly representative of the physiopathological processes of lymphomas.

To overcome the disadvantages of these 2D models, animal models have been developed, in which human cancer cells are grafted or injected. However, such animal models are expensive, difficult to reproduce and generally not representative of the physiological phenomena of human malignant haemopathies.

Recently, three-dimensional (3D) cancer cell cultures have been developed. These 3D cultures are particularly attractive for studying the mechanisms of cancer progression and better testing cancer treatments. Indeed, cells grown in 3D within a matrix or in aggregates have an architecture closer to tissue and tumour and show an expression of their genes similar to that of the tumour in vivo (Gravelle et al., 2014, Am. J. Pathol. 184:2082-295; Weiswald et al., 2009, Br. J. Cancer 101:473-482). In addition, 3D co-culture models mimicking cancer cell/stromal cell interactions make it possible to reproduce the tumour niche at least partially and to study its consequences on tumour progression or drug resistance.

With regard to the development of 3D lymphoma cultures, several techniques have been developed. 3D lymphoma models are generally obtained using collagen sponges (Kobayashi et al. 2010. Trends Immunol. 31:422-428), the so-called hanging drop technique (Gravelle et al. 2014. Am. J. Pathol. 184:282-295) or a polystyrene architecture (Caicedo-Carvajal et al. 2011. J. Tissue Eng 2011:362326). However, these techniques have many disadvantages, both in terms of cost and reproducibility, making them of little relevance for the industrial-scale study of new medicinal products. In addition, the 3D cultures developed do not integrate any element of the tumour microenvironment, thus limiting their relevance.

Thus, there remains a need for a 3D cell culture system that is representative pathophysiologically and/or in terms of mechanical properties of malignant haemopathies and can be produced on a large scale, without excessive cost.

### SUMMARY OF THE INVENTION

By working on the development of a 3D cell culture system that is most representative of lymphoma and of the biological and mechanical phenomena to which they are subjected in vivo, the inventors discovered that it is possible to fabricate in an automated and reproducible manner cellular microcompartments comprising at least malignant haematopoietic cells surrounded by an outer hydrogel capsule, using a coextrusion system. More precisely, the inventors discovered that by coextruding a hydrogel solution with a cell solution comprising lymphomatous cells, and optionally lymphoid stromal cells, i.e., close to the stromal cells that infiltrate lymphomas, and extracellular matrix, said cells aggregate within the hydrogel capsule to organize themselves into a cell mass approximating the cell organization within a lymphoma. In addition, depending on the type of cells coextruded with malignant haematopoietic cells, the inventors have discovered that it is possible to recreate a tumour niche mimicking a tumour niche in vivo. Thus, it is possible to obtain microcompartments in which the nature of the cells and the intercellular interactions are substantially similar to those observed in a lymphomatous or leukaemic tumour niche. The cellular microcompartments developed according to the invention are particularly relevant as 3D models of malignant haemopathies, in particular for screening and identifying new candidate molecules for the treatment of lymphomas and/or leukaemias. In addition, the process according to the invention makes it possible to obtain very large quantities of microcompartments of perfectly controlled dimensions. The microcompartments obtained are easy to handle, making them particularly suitable for large-scale use, particularly in the pharmaceutical field.

Therefore, a subject matter of the invention is a process for preparing a cellular microcompartment comprising an aggregate of cells containing malignant haematopoietic cells encapsulated in a hydrogel layer, according to which a hydrogel solution and a cell solution comprising malignant haematopoietic cells are coextruded concentrically, then crosslinked.

Advantageously, the cell solution comprises lymphomatous cells or leukaemic cells.

It is also possible to provide stromal cells and extracellular matrix in the cell solution in order to obtain a cellular microcompartment that approximates, in terms of cellular interactions and organization, a tumour niche.

Another subject matter of the invention is a cellular microcompartment obtainable by the process according to the invention, wherein said microcompartment comprises an aggregate of cells comprising at least malignant haematopoietic cells, encapsulated in a hydrogel layer.

According to the invention, a cellular microcompartment consists of a single aggregate of cells encapsulated in a hydrogel layer.

In an embodiment, said microcompartment comprises an aggregate of cells, consisting only of lymphomatous cells, encapsulated in a hydrogel layer. In another embodiment, the microcompartment comprises an aggregate of cells, consisting only of leukaemic cells, encapsulated in a hydrogel layer.

In another embodiment, said microcompartment comprises an aggregate of cells composed in particular of lymphomatous cells and lymphoid stromal cells, as well as an extracellular matrix layer between the aggregate of cells and the hydrogel layer.

In another embodiment, said microcompartment comprises an aggregate of cells composed in particular of leukaemic cells and medullary stromal cells, as well as an extracellular matrix layer between the aggregate of cells and the hydrogel layer.

The invention also relates to a cellular microcompartment comprising an aggregate of cells encapsulated in a hydrogel layer, wherein the aggregate of cells comprises malignant haematopoietic cells, such as lymphomatous cells or leukaemic cells, and stromal cells, said microcompartment further comprising an extracellular matrix layer between the aggregate of cells and the hydrogel layer.

Another subject matter of the invention is a method for screening or identifying a compound for the treatment and/or prevention of lymphoma, comprising the steps of:

(a) bringing a cellular microcompartment according to the invention, possibly without a hydrogel layer, into contact with a compound to be tested;

(b) selecting the compound capable of at least partially inhibiting the growth of the cell aggregate of said cellular microcompartment and/or at least partially killing cells of the cell aggregate of said cellular microcompartment.

Advantageously, such a screening method is performed in vitro.

Another subject matter of the invention is a use of a cellular microcompartment according to the invention for screening or identifying a compound for the treatment of malignant haemopathy, such as lymphoma or leukaemia.

Advantageously, such a use for screening is an in vitro use.

### BRIEF DESCRIPTION OF THE FIGURES

FIG. 1: Encapsulation of SUDHL4 and HLY1 lymphomatous cells in an alginate capsule. The SUDHL4 and HLY1 cells express GFP. FIG. 1A shows images of the capsules in phase contrast and fluorescence at different times (D1-D11) after encapsulation, the images being acquired with an Olympus CKX41 microscope (10× objective). FIG. 1B shows the growth curves measured for the cell clusters within the alginate capsules from the photos, using the ImageJ® software.

FIG. 2: Formation of cellular microcompartments according to the invention, comprising only lymphomatous or leukaemic cells (A) or lymphomatous or leukaemic cells, and stromal cells (B). 1: hydrogel capsule, 2: capsule lumen, 3: lymphomatous cells, 4: growth phase, 5: hydrogel capsule comprising a cluster of lymphomatous cells, 6: hydrogel capsule dissolution step, 7: lymphomatous cell cluster, 8: extracellular matrix layer, 9: stromal cells, 10: growth phase, 11: hydrogel capsule containing a cluster of lymphomatous and stromal cells, 12: cluster of lymphomatous and stromal cells.

FIG. 3: Cellular microcompartments comprising a cluster of DOHH2 follicular lymphomatous cells and lymphoid stromal cells (Resto, Arne-Thomas Blood 2007; 109:693) in an alginate capsule coated with an inner layer of Matrigel® at D9 after encapsulation. The images were obtained in phase contrast using a Leica DMI8 microscope (10× objective).

FIG. 4: Dissolution of the alginate capsule of cellular microcompartments comprising a cluster of DOHH2 follicular lymphomatous cells (A) and a cluster of DOHH2 follicular lymphomatous cells and Resto cells (B) at D9 after encapsulation.

FIG. 5: Flow cytometry analysis of dead cells in cell clusters comprising SUDHL4 lymphomatous cells (5A) or HLY1 lymphomatous cells (B) after different encapsulation times.

FIG. 6: Analysis of the additional effect of the stromal tumour niche on lymphomatous cell growth. Images of cellular microcompartments in an alginate capsule covered with an inner layer of Matrigel® comprising only Resto cells (A), only DOHH2 lymphomatous cells (B), Resto cells and DOHH2 lymphomatous cells (C). The images were obtained in phase contrast using a Leica DMI8 microscope (10× objective). The scale bar is the same for all three images.

FIG. 7: Comparative effects of etoposide (A) and cisplatin (B) on cell death after bringing into contact, for 48 h, a suspension of HLY1 cells (suspension) or a cluster of HLY1 cells from a cellular microcompartment according to the invention, comprising only lymphomatous cells, with increasing doses of etoposide (μg/mL) or cisplatin (μM).

FIG. 8: Cellular microcompartment showing the differentiation of stromal cells into pro-tumour lymphoid stroma. Cellular microcompartments comprising a cluster of DOHH2 follicular lymphomatous cells and Resto lymphoid stromal cells in an alginate capsule coated with an inner layer of Matrigel® at D8 after encapsulation. CD20 (revealing B lymphocytes), GFP (revealing Resto cells) and TG2 staining was performed on a fixed and paraffin-embedded spheroid. The image was taken under an LSM510 confocal microscope, 20× objective. The image on the right is a superposition of the first three images. The arrows indicate Resto (GFP+) cells expressing TG2.

FIG. 9: Study of the diffusion of doxorubicin in SUDHL4 cells grown in suspension or in spheroids. SUDHL4 cells grown in suspension or after 7 days in 3D with or without extracellular matrix (ECM) (Mg: Matrigel) and Resto cells are treated for 24 h with doxorubicin (1 μM) . After dissociation of the 3D-grown cells, the fluorescence intensity of the cells was analysed in flow cytometry.

FIG. 10: Representative images showing the penetration of antibodies into the cellular microcompartment. A) Staining with anti-CD19-PE AB. Aa) Microcompartment containing DOHH2 follicular lymphomatous cells grown in 3D in the presence of ECM and Resto stromal cells (SC-GFP+). Ab) Microcompartment containing DLBCL SUDHL4 cells grown in 3D in the presence of ECM. B) Staining with rituximab (RTX)-633. Ba) Microcompartment containing DOHH2 follicular lymphomatous cells grown in 3D in the presence of ECM and Resto stromal cells (SC-GFP+) visualized in Bb). The capsules are incubated for 12 h with the ABs, then imaged with a Zeiss LSM510 confocal microscope with a 25× objective. The nuclei are stained blue with DAPI. The outline of the capsules is visible (line at the periphery on the images).

FIG. 11: Comparison of the cytotoxic effects of doxorubicin and of etoposide on DLBCL SUDHL4 cells grown in 2D (suspension) or in 3D ± ECM ± Resto. Cells or spheroids at D7 post-encapsulation are treated for 48 h with drugs. At the end of the treatment, cell viability is measured using the CellTiter-Glo® 3D Cell Viability Assay Kit (Promega).

FIG. 12: Formation of cellular microcompartments according to the invention as a function of time from T lymphocytes in primary culture from patients with Sezary lymphoma. At different culture times after encapsulation, the cells are stained with calcein-AM to visualize live cells and with propidium iodide (PI) to visualize dead cells. The nuclei are stained with DAPI. An increase in cell death is noted when the spheroid is very dense, 10 days after encapsulation (D10).

### DETAILED DESCRIPTION

### Cellular Microcompartment

A subject matter of the invention is a 3D cellular microcompartment comprising an aggregate, or cluster, of malignant haematopoietic cells encapsulated in a hydrogel shell.

In the context of the invention, the terms “hydrogel layer”, “hydrogel capsule” or “hydrogel shell” refer to a three-dimensional structure formed from a matrix of polymer chains swollen by a liquid, preferentially water. Advantageously, the one or more polymers in the hydrogel layer are polymers that can be crosslinked when subjected to a stimulus, such as temperature, pH, ions, etc. Advantageously, the hydrogel used is biocompatible, in the sense that it is not toxic to cells. In addition, the hydrogel layer must allow the diffusion of oxygen and nutrients to feed the cells contained in the cellular microcompartment and allow them to survive. Advantageously, the hydrogel layer also allows molecules to pass through for testing, such as pharmaceutical molecules. The polymers in the hydrogel layer can be of natural or synthetic origin. For example, the outer hydrogel layer contains one or more polymers among sulfonate polymers, such as sodium polystyrene sulfonate, acrylate polymers, such as sodium polyacrylate, polyethylene glycol diacrylate, the compound gelatin methacrylate, polysaccharides, and in particular polysaccharides of bacterial origin, such as gellan gum, or of vegetable origin, such as pectin or alginate. In an embodiment, the outer hydrogel layer comprises at least alginate. Preferably, the outer hydrogel layer comprises only alginate. In the context of the invention, “alginate” refers to linear polysaccharides formed from δ-D-mannuronate (M) and α-L-guluronate (G), salts and derivatives thereof. Advantageously, the alginate is a sodium alginate, composed of more than 80% G and less than 20% M, with an average molecular weight of 100 to 400 kDa (e.g., PRONOVA® SLG100) and a total concentration between 0.5% and 5% by density (weight/volume).

Advantageously, the hydrogel layer comprises cell-repellent polymers, such as natural polysaccharides (for example sodium alginate), or polymers comprising polyethylene glycol units, in order to facilitate, if necessary, the separation of said hydrogel layer from the cell aggregate it envelops or its degradation without affecting the structure of the cell aggregate.

The cellular compartment according to the invention is characterized by the presence, in the internal volume of the hydrogel shell, of an aggregate of cells organized in a cohesive cluster within which the cells interact.

According to the invention, the cell aggregate comprises malignant haematopoietic cells. The term “malignant haematopoietic cells” refers to cancer cells resulting from the differentiation of lymphoid progenitors (i.e., lymphocytes) or myeloid progenitors (i.e., erythrocytes, leukocytes, platelets). Preferentially, in the context of the invention, the malignant haematopoietic cells are selected from lymphomatous cells and leukaemic cells.

According to a particular embodiment of the invention, the cell aggregate contained in the outer hydrogel shell comprises lymphomatous cells. In the context of the invention, “lymphomatous cells” refers to malignant lymphoid cells. Advantageously, the lymphomatous cells are selected from follicular lymphoma, diffuse large B-cell lymphoma, Burkitt lymphoma, mantle cell lymphoma, peripheral T-cell lymphoma, lymphoblastic lymphoma, anaplastic lymphoma, marginal zone lymphoma, lymphoma of mucosa-associated lymphoid tissue (MALT), lymphoplasmacytic lymphoma, and/or lymphoma of the spleen, cutaneous T-cell lymphoma, cutaneous B-cell lymphoma.

According to another specific embodiment of the invention, the cell aggregate contained in the outer hydrogel shell comprises leukaemic cells. In the context of the invention, “leukaemic cells” refers to malignant blood cells. In particular, the leukaemic cells can be selected from acute myeloblastic leukaemia cells, chronic myeloid leukaemias, chronic lymphoid leukaemias, acute leukaemias.

According to the invention, the malignant haematopoietic cells can be derived from cellular models but may also be obtained from patients. The use of lymphomatous or leukaemic cells from a particular patient may be of particular interest in the context of personalized medicine, for identifying one or more molecules particularly suitable for the treatment of lymphoma or leukaemia in said patient. In this case, the patient's tumour cells are advantageously purified before use. Advantageously, purification is done by negative selection, in order to avoid the introduction of antibodies into the cell culture.

In one embodiment, the cell aggregate of the cellular microcompartment comprises only lymphomatous cells. The organization of lymphomatous cells into a cohesive cell cluster within the hydrogel capsule gives said cells a resistance to the penetration of external molecules that approximates the resistance observed within cells of a lymphoma.

In another embodiment, the cell aggregate of the cellular microcompartment comprises only leukaemic cells.

In another embodiment, and in order to approximate tumour niches histologically, the cell aggregate comprises, in addition to malignant haematopoietic cells, stromal cells. The nature of the stromal cells chosen depends advantageously on the nature of the associated malignant haematopoietic cells. In this case, the microcompartment further comprises an extracellular matrix layer. Indeed, the inventors have shown that the presence of an extracellular matrix is necessary for the adhesion and survival of stromal cells and for the formation of the mixed cell aggregate within the hydrogel capsule. According to the invention, the extracellular matrix layer covers advantageously the inner surface of the hydrogel shell. The extracellular matrix layer comprises a mixture of proteins and of extracellular compounds that promote cell culture, particularly that of stromal cells. Preferentially, the extracellular matrix comprises structural proteins, such as laminins containing the α1, α4 or α5 subunits, the β1 or β2 subunits, and the γ1 or γ3 subunits, entactin, vitronectin, fibronectin, laminins, collagen, as well as growth factors, such as TGF-beta and/or EGF. In an embodiment, the extracellular matrix layer consists of, or contains, Matrigel® and/or Geltrex®.

In a particular embodiment, the malignant haematopoietic cells are lymphomatous cells and the stromal cells are lymphoid stromal cells, such as adipose-derived stem cells (ADSC), or more specifically, Resto cells. Resto cells are defined as tonsil-derived stromal cells. The isolation and characterization of Resto cells can be done according to the protocol described in the publication Arne-Thomas et al. Blood 2007. The tonsils are cut into pieces and incubated in a solution containing DNase I and collagenase IV. The cell suspension is then deposited on a Percoll® gradient. The cells at the interface of the 15%/25% Percoll® fraction are cultured. After 48 hours of culture, the stromal cells (Resto) adhere to the plastic and the suspended cells are removed. Resto cells are characterized by a fusiform morphology and by the absence of haematopoietic cell markers (CD45) and the presence of mesenchymal cell markers (CD105, CD90 and CD73). These cells are also characterized by their potential for differentiation into adipocytic, chondroblastic and osteoblastic lineages. Resto cells are known to have characteristics similar to those of reticular fibroblastic cells in secondary lymphoid organs: secretion of chemokines, fibronectin and a transglutaminase network in response to tumour necrosis factor (TNF)-α and lymphotoxin (LT)-α1β2.

More generally, the aggregate of cell may comprise, in addition to lymphomatous cells, cells normally present in the microenvironment of a lymphoma. In the context of the invention, “microenvironmental cells” refers to the cells present in the aggregate of cells that are not lymphomatous cells. Thus, according to the invention, the cell aggregate may also comprise, in addition to stromal cells, immune system cells such as macrophages.

In a particular embodiment, the malignant haematopoietic cells are leukaemic cells and the stromal cells are medullary stromal cells, such as HS-5 cells, or bone marrow mesenchymal stromal cells (MSC).

Advantageously, the ratio of lymphomatous cells to stromal cells in the cell aggregate is between 1:1 and 1000:1. In the case of lymphomatous cells derived from cell lines, the ratio may vary over time, with the amount of lymphomatous cells tending to increase exponentially compared with stromal cells. In general, once the microcompartment has reached its maximum size, usually within eight days of encapsulation of the cells in the hydrogel shell (D8), and the cells can no longer grow inside the hydrogel shell, the ratio of lymphomatous cells to stromal cells is between 1:1 and 10,000:1.

In one embodiment, the cell density in the cellular microcompartment at D8 is between one hundred and several thousand cells. For example, a microcompartment with a diameter of 200 um preferably contains 100 to 10,000 cells.

Preferentially, the cellular microcompartment is closed. It is the outer hydrogel layer that gives the cellular microcompartment its size and shape. The microcompartment can have any shape compatible with cell encapsulation, particularly spheroid, ovoid or tubular shape. The cell aggregate is constrained in the internal volume of said hydrogel layer, and once the cells reach confluence, the aggregate can no longer increase in volume.

In a particular embodiment, the cellular microcompartment has a diameter or a smallest dimension between 50 μm and 600 μm. The term “smallest dimension” means twice the minimum distance between a point on the outer surface of the hydrogel layer and the centre of the microcompartment. Advantageously, the thickness of the outer hydrogel layer represents 5% to 30% of the radius of the microcompartment. In the context of the invention, the “thickness” of a layer is the dimension of said layer extending radially from the centre of the microcompartment.

In a particular exemplary embodiment, the cellular microcompartment has a diameter or a smallest dimension between 50 μm and 300 μm. Such dimensions ensure that all cells in the cell aggregate, including those in the centre of said cell aggregate, have sufficient access to oxygen and nutrients that diffuse through the hydrogel layer. Thus, no hypoxia and/or necrosis is observed within such a microcompartment, all cells having sufficient access to the small molecules that diffuse through the hydrogel shell.

In another exemplary embodiment, the cellular microcompartment has a diameter or a smallest dimension between 500 μm and 600 μm. In this case, the cells in the centre of the cell aggregate have little or no access to oxygen and nutrients that diffuse through the hydrogel shell. Such microcompartments are particularly attractive for the study of hypoxia and/or necrosis that can sometimes occur in lymphoma.

Processes for Preparing Cellular Microcompartments

Another subject matter of the invention relates to processes for preparing cellular microcompartments to obtain cellular microcompartments comprising an aggregate of cells containing malignant haematopoietic cells encapsulated in an outer hydrogel shell. After encapsulation of the cells, they will reorganize within the hydrogel shell to form a cohesive cluster. Encapsulation is carried out by means of a concentric coextrusion process, in which the hydrogel solution is coextruded with the cell solution, before being crosslinked by means of a crosslinking solution capable of crosslinking the hydrogel. The term “concentric coextrusion” means that the solutions are coextruded in such a way that one solution surrounds the other. In this case, the concentric coextrusion is such that the hydrogel solution surrounds the cell solution. In an embodiment, drops of coextruded solutions then fall into the crosslinking solution, comprising a crosslinking agent capable of crosslinking the hydrogel and thus forming a hydrogel capsule around the cells. In another embodiment, the solutions are coextruded directly into the crosslinking solution to form an outer hydrogel tube in which the cells will be organized. In another embodiment, drops of coextruded solutions pass through a crosslinking aerosol (made from a crosslinking solution) to allow at least partial crosslinking of the hydrogel layer around the drop of cell solution. Advantageously, the partially crosslinked microcompartments then fall into a crosslinking solution where the crosslinking ends.

Any extrusion process that allows hydrogel and cells to be coextruded concentrically can be used. In particular, it is possible to produce cellular microcompartments according to the invention by adapting the method and the microfluidic device described in Alessandri et al., (PNAS, Sep. 10, 2013 vol. 110 no. 37 14843-14848; Lab on a Chip, 2016, vol. 16, no. 9, pp. 1593-1604) or in Onoe et al., (Nat Material 2013, 12(6):584-90). For example, the process according to the invention is implemented by means of a concentric double-wall extrusion device as described in patent FR2986165.

In the context of the invention, “crosslinking solution” means a solution comprising at least one crosslinking agent adapted to crosslink a hydrogel comprising at least one hydrophilic polymer, such as alginate, when brought into contact with it. The crosslinking solution can be, for example, a solution comprising at least one divalent cation. The crosslinking solution may also be a solution comprising another known crosslinking agent of the alginate or of the hydrophilic polymer to be crosslinked, or a solvent, for example water or an alcohol, adapted to allow crosslinking by irradiation or by any other technique known in the art. Advantageously, the crosslinking solution is a solution comprising at least one divalent cation. Preferentially, the divalent cation is a cation used to crosslink alginate in solution. For example, it may be a divalent cation selected from the group consisting of Ca²⁺, Ba²⁺, Ba²⁺ and Sr²⁺, or a mixture of at least two of these divalent cations. The divalent cation, for example Ca²⁺, can be combined with a counterion to form for example Cacl₂ or CaCO₃ solutions, well known to the skilled person. The crosslinking solution may also be a solution comprising CaCO₃ coupled to glucono delta-lactone (GDL) forming a CaCO₃-GDL solution. The crosslinking solution can also be a CaCO₃-CaSO₄-GDL mixture. In a particular embodiment of the process according to the invention, the crosslinking solution is a solution comprising calcium, in particular in the Ca²⁺ form. The crosslinking solution may also be a solution comprising polylysine. The skilled person is able to adjust the nature of the divalent cation and/or of the counterion, as well as its concentration, to the other parameters of the process of the present invention, in particular to the nature of the polymer used and to the desired speed and/or degree of crosslinking. For example, the concentration of divalent cation in the crosslinking solution is between 10 and 1000 mM. The crosslinking solution may comprise components, well known to the skilled person, other than those described above, to improve the crosslinking of the hydrogel sheath under specific conditions, including time and/or temperature.

According to the invention, the hydrogel solution is coextruded with a cell solution.

Advantageously, the cell density in the cell solution is between 1·10⁶ and 100·10⁶ cells/mL. In a particular embodiment, the cell solution used for coextrusion contains only lymphomatous cells suspended in culture medium.

In a particular embodiment, the cell solution used for coextrusion contains only leukaemic cells suspended in culture medium.

In another embodiment, the cell solution used for coextrusion includes lymphomatous cells and lymphoid stromal cells, suspended in an extracellular matrix. Advantageously, the number ratio of lymphomatous cells to stromal cells in the cell solution is between 1:1 and 1:2. Optionally, such a solution may also include immune cells, preferentially selected from macrophages. In this case, the number ratio of lymphomatous cells to microenvironmental cells in the cell solution is advantageously between 1:1 and 1:2.

In another embodiment, the cell solution used for coextrusion comprises leukaemic cells and medullary stromal cells, suspended in an extracellular matrix. Advantageously, the number ratio of leukaemic cells to stromal cells in the cell solution is between 1:1 and 1:2.

When the cell solution comprises extracellular matrix, the cell suspension advantageously represents between 50% and 95% of the volume of the solution, while the extracellular matrix represents between 5% and 50% of said volume.

In a particular embodiment, coextrusion also involves an intermediate solution, comprising sorbitol. In this case, coextrusion is carried out in such a way that the intermediate solution is extruded between the hydrogel solution and the cell solution.

In a particular embodiment, the extrusion rate of the hydrogel solution is between 5 and 100 mL/h, preferentially between 15 and 60 mL/h.

In a particular embodiment, the extrusion rate of the cell solution is between 5 and 100 mL/h, preferentially between 10 and 50 mL/h.

In a particular embodiment, the extrusion rate of the intermediate solution is between 5 and 100 mL/h, preferentially between 10 and 50 mL/h.

The coextrusion rate of the different solutions can be easily adjusted by the skilled person, in order to adapt the diameter or the smallest dimension of the cellular microcompartment and/or the thickness of the hydrogel layer. Preferentially, the extrusion rates of the cell solution and the intermediate solution are identical. Advantageously, the extrusion rate of the hydrogel solution is substantially equal to the extrusion rate of the cell solution and possibly of the intermediate solution.

In a particular embodiment of the process according to the invention, the hydrogel solution, the intermediate solution and the cell solution are loaded into three concentric compartments of a coextrusion device, so that the hydrogel solution, forming the first flow, surrounds the intermediate solution that forms the second flow, which itself surrounds the cell solution that forms the third flow. The tip of the extrusion device, through which the three flows exit, opens above the crosslinking solution. For example, the tip of the extrusion device is about 50 cm, ± 10 cm, from the crosslinking solution. An electric field is generated at the outlet of the coextrusion device to allow the formation of microdroplets. For this purpose, for example, a copper ring is placed about 1 cm from the outlet of the coextrusion device. Microdroplets fall sequentially into the crosslinking bath where the hydrogel layer is crosslinked, forming an outer shell around the cells. Alternatively or additionally, the tip of the extrusion device opens into a crosslinking aerosol, formed by microdrops of crosslinking solution, so that the hydrogel layer of the microdroplets begins to crosslink in contact with the microdrops of the aerosol. Crosslinking may, if need be, continue in a crosslinking solution in which the microdroplets are received.

In order to improve the polydispersity of the drops at the outlet of the coextrusion device, and thus prevent the microdroplets from merging before reaching the crosslinking solution, a potential of +1 to +5 kV, and in particular a potential of +2 kV, can be applied to the hydrogel solution, for example by means of an electrode disposed in the hydrogel solution.

The process according to the invention makes it possible to very quickly obtain several thousand microcompartments that are substantially identical in terms of size and composition.

The process according to the invention makes it possible to encapsulate malignant haematopoietic cells, such as lymphomatous cells, in an outer hydrogel shell. After only a few hours, the cells contained in the hydrogel shell reorganize, so as to aggregate and form a cluster of cells that becomes cohesive after a few days.

Advantageously, the cellular microcompartment obtained by coextrusion is maintained in a suitable culture medium for two to twelve days before being used, preferentially between four and ten days. This latency time advantageously allows the cells to aggregate and form a cell cluster that mimics the cluster of cells within a lymphoma.

According to the invention, it is possible to use the cellular microcompartment obtained by coextrusion as it is, i.e., with the outer hydrogel shell. It is otherwise possible to hydrolyse the hydrogel shell prior to any use, in order to recover the cell aggregate. It is also possible to freeze the cellular microcompartment obtained by coextrusion (with the hydrogel shell) for subsequent use.

Applications

The cellular microcompartments according to the invention can be used for many applications, particularly for pharmacological purposes.

The cellular microcompartments according to the invention can be used for identification and/or validation tests of candidate molecules having an action on malignant haemopathies.

Depending on the malignant haematopoietic cells contained in the cellular microcompartment, it is possible to target a particular type of malignant haemopathy.

According to the invention, it is possible to use cellular microcompartments directly, i.e., with the outer hydrogel shell. In particular, the permeability of some hydrogels, such as alginate, is sufficient to allow molecules with a molecular weight of 200 kDa or less to pass through. It is therefore possible to study these molecules directly on the cellular microcompartments. In the case of molecules with a higher molecular weight, it is possible to hydrolyse the outer shell of the hydrogel before performing the tests. Thus, the study is done directly on the cell cluster. Advantageously, the hydrolysis of the hydrogel shell is carried out 6 or 10 days after coextrusion, so as to ensure that the cell cluster has formed properly and that the cells are cohesive.

The cellular microcompartments according to the invention can also be used in personalized medicine, using cells from a subject with lymphoma or leukaemia, to specifically test the reaction of said subject to different treatments, before selecting the most suitable treatment for said subject.

### EXAMPLES

### Example 1

### Process for Obtaining Cellular Microcompartments

Materials and Methods

Cells:

Human diffuse large B-cell lymphoma (DLBCL) cell lines (SUDHL4 and HLY1)

Human follicular lymphoma cell lines (DOHH2)

B lymphocytes from patient biopsies purified by negative selection (Maby-El Hajjami et al. Can Res 2009: 69 (7) 3228:37)

“Resto” stromal cells (Amé-Thomas et al. (Blood 2007))

Solutions:

Crosslinking solution: 100 mM CaCl₂ at 37° C.

Intermediate solution: 300 mM sorbitol

Hydrogel solution: 2.5% w/v alginate (LF200FTS) in 0.5 mM SDS

Extracellular matrix: Classic Matrigel® (without phenol red and with growth factors)

Coextrusion Device

3 Hamilton 12 ml syringes containing respectively sterile 2.5% alginate and the other two sterile 300 mM sorbitol

standard Teflon tubing, diameter 13 mm

neMESYS® syringe pump (CETONI) and associated software

3D printed injection chip (see publication Alessandri K et al., 2016)

Coextrusion Process

Alginate capsules are obtained according to the process described in Alessandri et al. (PNAS 2013, DOI:10.1073/pnas.1309482110 and LOC 2016 DOI:10.1039/c61c00133e) and in application WO2013113855.

More precisely, approximately 1·10⁶ cells are resuspended in a solution of sorbitol and Matrigel® to obtain a final volume of 100 μL with 50 vol % Matrigel®. This cell solution is stored at 4° C.

The microfluidic coextrusion device for the production of capsules is placed approximately 50 cm above a Petri dish containing the crosslinking solution.

The alginate solution, the sorbitol solution and the cell solution are then co-injected into the microfluidic coextrusion device to form composite droplets that are crosslinked as they fall into the calcium bath. The coextrusion device is operated for 10 seconds, and produces about 5000 alginate capsules per second, for a total of about 50,000 capsules. The alginate capsules are then recovered by filtering the calcium bath with a 40 μm mesh cell strainer that retains the capsules. The latter are rinsed with medium base and then resuspended in the final medium.

To improve polydispersity, a potential of +2 kV was applied via an electrode in the alginate. A 3 cm diameter grounded copper ring is positioned approximately 1 cm from the tip of the microfluidic coextrusion device to generate the electric field necessary for electroforming droplets.

A] Cellular Microcompartments Comprising Only SUDHL4 or HLY1 Lymphomatous Cells

Before encapsulation, lymphomatous cells are cultured in DMEM with 10% foetal calf serum (FCS) in a humid atmosphere at 37° C. in the presence of 5% CO₂.

At the time of encapsulation, the cells are centrifuged and resuspended in sorbitol (300 mM) at a rate of 10·10⁶ to 100·10⁶ cells/mL.

After encapsulation, the number of cells per capsule varies between 30 and 100. The capsules are cultured in a DMEM with 10% FCS added in a CO₂ oven at 37° C. The medium is changed every 2 to 3 days.

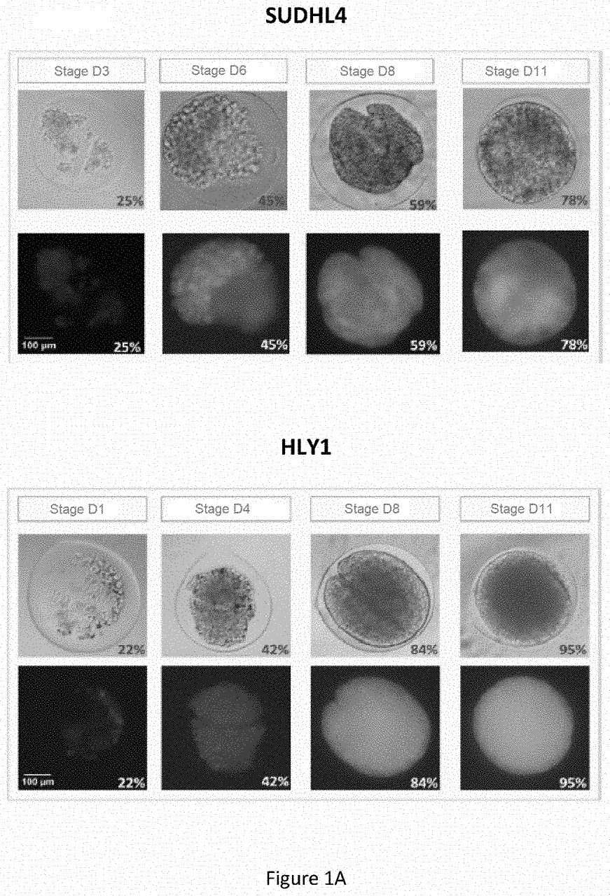

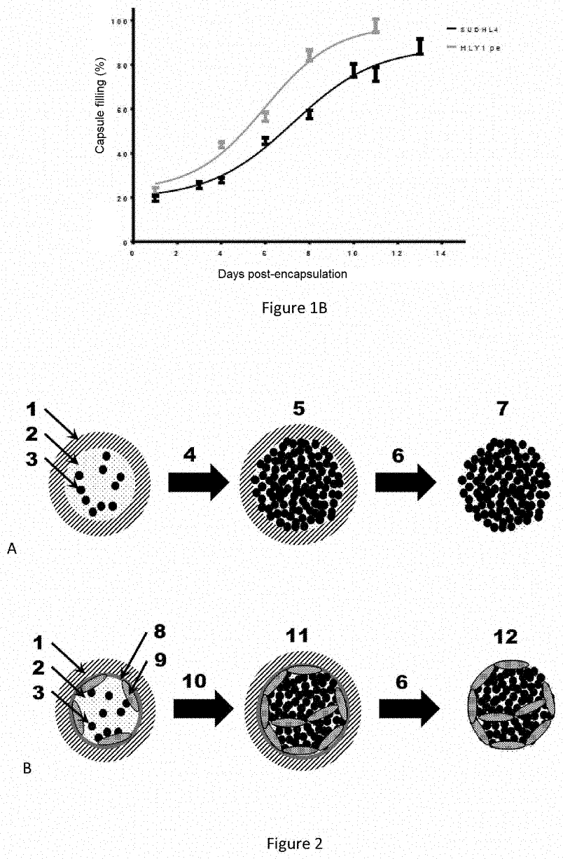

To quantify the growth of cell clusters, the capsules are divided into 96-well plates, one capsule per well. The cell clusters are then imaged regularly (every 2 days) in phase contrast and fluorescence if the cell line expresses a fluorescent protein. This results in a series of photos representing the growth of a unique cluster of cells over time. From these photos, the area of the cell clusters is measured using the ImageJ® software and growth curves are established (FIG. 1B).

It is thus observed that the cell clusters are formed between D4-D10 after encapsulation. More precisely, between D6 and D10, it is observed that the cells aggregate and form a single mass in the alginate capsule (FIG. 1A).

B] Cellular Microcompartments Comprising Lymphomatous Cells and Stromal Cells

In order to approximate the microenvironment of a lymph node, lymphomatous cells (DOHH2) are co-cultured with the extracellular matrix Matrigel® and “Resto” stromal cells.

The Resto cells were first cultured in DMEM supplemented with 10% foetal calf serum in a humid atmosphere at 37° C. with 5% CO₂. At the time of encapsulation, the cells are detached from the support by action of trypsin, then they are resuspended in the extracellular matrix in the presence of lymphomatous cells at a 1:1 ratio.

The cell density of Resto cells and lymphomatous cells can vary from 10·50·10⁶ cells/mL.

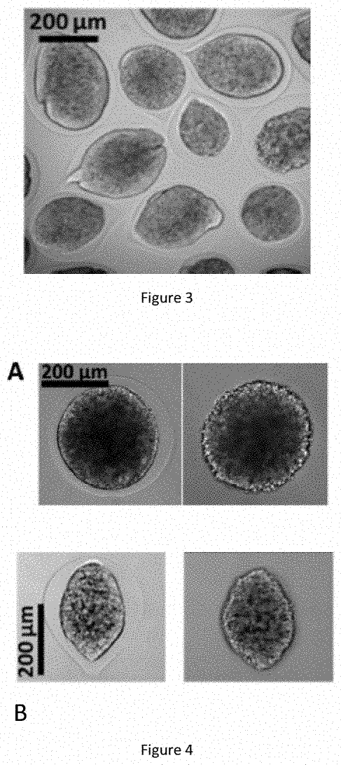

The coextrusion encapsulation method makes it possible to obtain a coating of the inner wall of the alginate capsules with the extracellular matrix. This coating allows the adhesion of stromal cells and promotes the formation of the tumour niche. Within a few days, the cells organize themselves freely and form a cluster of cohesive cells after 4-10 days of culture (FIG. 3).

C] Cellular Microcompartments Produced from Patient Cells

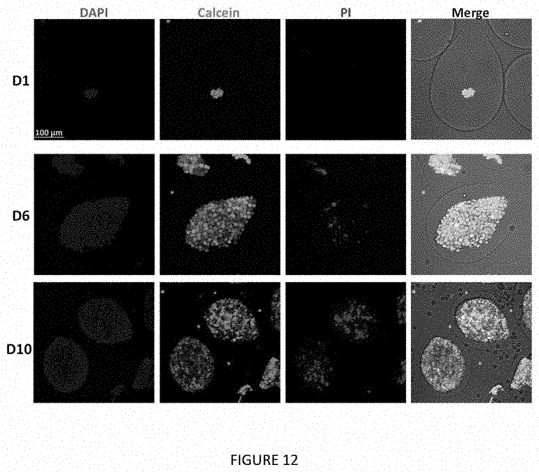

Microcompartments were produced according to the invention from lymphocyte cells from patients with Sézary syndrome (leukaemic form of cutaneous T-cell lymphoma). After encapsulation, the cells grow inside the alginate capsules and form a cluster of cells after about ten days (FIG. 12). This confirms that the process according to the invention allows cellular microcompartments to be obtained from patients' primary cells, which suggests the use of this process in personalized medicine.

### Example 2

### Analysis of Cellular Microcompartments

A] Real-Time Imaging

Capsules containing clusters of SUDHL4 or HLY1 cells are placed in agarose wells covered with DMEM without phenol red (at D1-D6). Image acquisition is done with the confocal microscope (Zeiss LSM 510). Two types of analyses are performed: an analysis at a time t after labelling the microcompartments with a fluorophore, and an analysis in intermittent imaging, after optionally labelling cells with a fluorophore. The cells are stained with calcein-AM and propidium iodide to visualize dead cells; the cell nuclei are stained blue with Hoechst 33342.

It is thus possible to monitor the migration of cells within the capsule, as well as the interactions between cells.

B] Paraffin Section Immunofluorescence

To be able to study the expression or regionalization of proteins in cells within cell clusters, the paraffin section immunofluorescence technique was adapted for capsule analysis: capsules containing cell clusters are collected and included in a 2% low-melting-temperature agarose gel. Once gelled, the agarose block containing the capsules is immersed in a 4% paraformaldehyde fixative for 30 min. After fixation, the samples are treated according to the protocols conventionally described for immunohistofluorescence.

The expression of the Ki67 protein, which is a marker of cell activation, and the expression of cleaved caspase-3, which is used to assess cell death, were evaluated. Staining was carried out on cell cluster sections roughly corresponding to the centre of the clusters.

No regionalization of cell death or proliferation is observed within cell clusters. These observations are quite comparable to those made in SUDHL4 cell tumours obtained after xenotransplantation into immunodeficient mice.

C] Analysis of Extracellular Matrix in Cell Clusters

One of the characteristics of the lymph node microenvironment is the presence of extracellular matrix secreted by stromal cells and tumour B lymphocytes. The extracellular matrix consists mainly of fibronectin, collagen I and laminin.

An immunofluorescence analysis showed the presence of these three types of matrix in the cell clusters of the microcompartments, while the same cells grown in suspension do not express these matrices.

D] Dissolution of Alginate Capsules

For high-throughput qualitative and quantitative analysis of cell clusters, it is important to be able to recover the cells contained in the hydrogel capsule. Once recovered, they can be sorted and/or analysed by flow cytometry or by any other appropriate analytical method, such as high-throughput sequencing, biochemical assays, etc.

Microcompartments (at D9 after encapsulation) are collected and the alginate capsule is dissolved by dipping the microcompartments in a phosphate buffer with 1 mM EGTA added. FIG. 4 shows an example of dissolution of a capsule containing a cluster of cells comprising only SUDHL4 lymphomatous cells (FIG. 4A) and a capsule containing a cluster of cells comprising DOHH2 lymphomatous cells and stromal cells (FIG. 4B).

It can be seen that after dissolution of the capsule, the cells constituting the cell cluster do not disperse and show a cohesion that is compatible with the presence of extracellular matrix.

E] Flow Cytometry

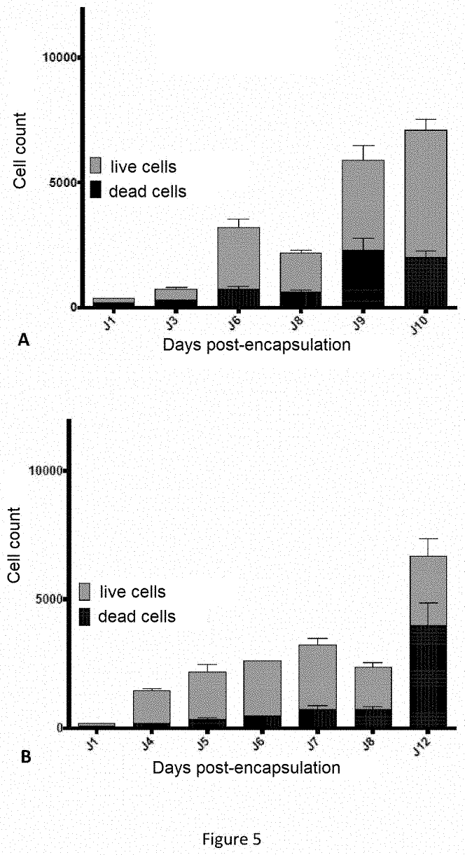

In order to access the cell count within the cell clusters and the percentage of dead cells at the different growth stages, the alginate capsules are dissolved and the cell clusters dissociated before being incubated with a marker of apoptosis (TMRM) and then analysed by flow cytometer in the presence of counting beads (FIG. 5).

Over time, it is observed that the cell count increases, while the proportions of dead cells within the cell clusters decrease between D3 and D10, which is in correlation with the increase in the volume of the spheroid described in FIG. 1A.

F] Additional Effect of the Tumour Niche on Lymphomatous Cell Growth

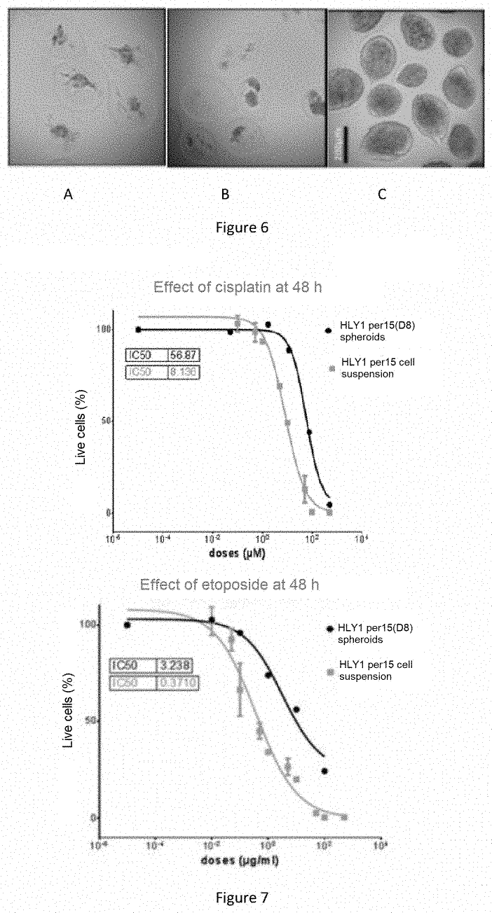

Tumour B lymphocytes from patients, as well as certain cell lines, are not able to survive and/or form cell clusters when cultured alone. As can be seen in FIG. 6 (A-C), the reconstitution of a tumour niche similar to that found in the lymph node (C: Resto+DOHH2 cells) promotes the growth of tumour B lymphocytes.

G] Differentiation of Stromal Cells into Pro-Tumour Lymphoid Stroma

During the formation of the follicular lymphoma tumour niche, stromal cells differentiate into pro-tumour lymphoid stroma (Thomazy et al., 2003; Ohe et al., 2016) expressing, among others, transglutaminase 2 (TG2) which has a role in extracellular matrix stabilisation and cell adhesion.

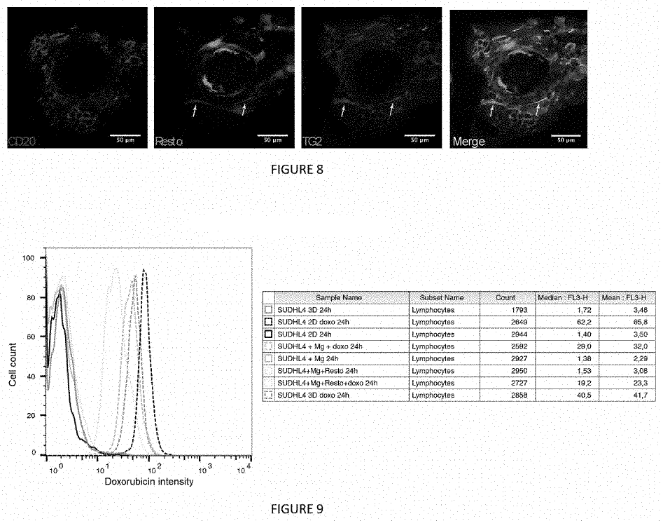

As can be seen in FIG. 8, the culture of Resto stromal cells (FRC) in the presence of tumour B cells and extracellular matrix results in the expression of TG2, revealed by immunostaining. This shows that the 3D cell culture model according to the invention reproduces the main features of a lymphoma tumour niche, namely the additional effect of the stroma on B lymphocytes and the differentiation of the stroma into pro-tumour stroma.

### Example 3

### Screening of Anti-Cancer Molecules

It is recognized that the chemoresistance of certain lymphomas is partly related to poor penetration of drugs into 3D structures and to the presence of microenvironmental cells.

The efficacy of two conventional chemotherapies (cisplatin and etoposide) was tested in parallel on cells grown in suspension (2D) and on cell clusters from cellular microcompartments according to the invention (3D), treated with increasing concentrations of these molecules. After 48 h of incubation, the cells are stained with TMRM (the alginate capsules are first dissolved and the cell clusters dissociated) and then analysed with a cytometer to evaluate the percentage of cell death for each dose of the molecule.

The results show that the cells organized in cell clusters (3D), which have a structure closer to the structure of a tumour, are less sensitive to conventional chemotherapies than the same cells grown in suspension (FIG. 7). This confirms that the 3D model according to the invention is more relevant for screening anti-cancer drugs than suspended cells.

### Example 4

### Drug Diffusion in the Cellular Microcompartment

A] Conventional Chemotherapies

Preclinical cancer drug discovery has the worst success rate of all therapeutic trials, with less than 5% of candidate compounds entering Phase III clinical trials. One explanation for these failures is the lack of a relevant model capable of reproducing the diffusion of drugs within a tumour. Indeed, one hypothesis to explain the decrease in the efficacy of molecules during the transition from pre-clinical tests to clinical trials would be that the cell density within tumours would slow down the penetration and the diffusion of drugs, thereby decreasing their efficacy.

As shown in Example 3 above, the 3D model by invention is relevant for studying the diffusion of drugs within a lymphoma tumour niche.

For drug diffusion tests, the auto-fluorescent properties of doxorubicin (which is also used in standard treatment of B-cell lymphomas) were used.

Thus, SUDHL4 cells grown in suspension or in 3D with or without extracellular matrix (ECM) or Resto stromal cells were incubated for 24 h with or without doxorubicin (1 μM).

The fluorescence intensity of the cells was then measured by flow cytometry.

The results show that the cells grown in 3D in the microcompartment according to the invention are less fluorescent than the cells grown in 2D, suggesting that in a 3D context, the cells are less exposed to chemotherapy. Very interestingly, it is observed that the diffusion of the drug is slowed further when the cells are grown in 3D in the presence of matrix and Resto cells (FIG. 9). Thus, by slowing the diffusion of molecules within spheroids according to the invention (and by analogy within tumours), it is shown how the microenvironment can play a role in drug resistance.

B] Antibodies

The standard treatment for B-cell lymphomas consists of conventional polychemotherapy combined with immunotherapy against CD20 expressed on the surface of mature B lymphocytes.

To be able to test the therapeutic potential of certain immunotherapies in the 3D cell culture system according to the invention, it is of interest to show that antibodies (AB) are able to penetrate the microcompartment.

To test this, the spheroids were incubated at D7-D9 post-encapsulation in the presence of

anti-CD19 AB directly coupled to phycoerythrin (PE) (very strongly expressed in B lymphocytes), or

rituximab, which is the therapeutic AB used for B-cell lymphomas, which has been directly coupled with a fluorophore (λₑₓ=633 nm).

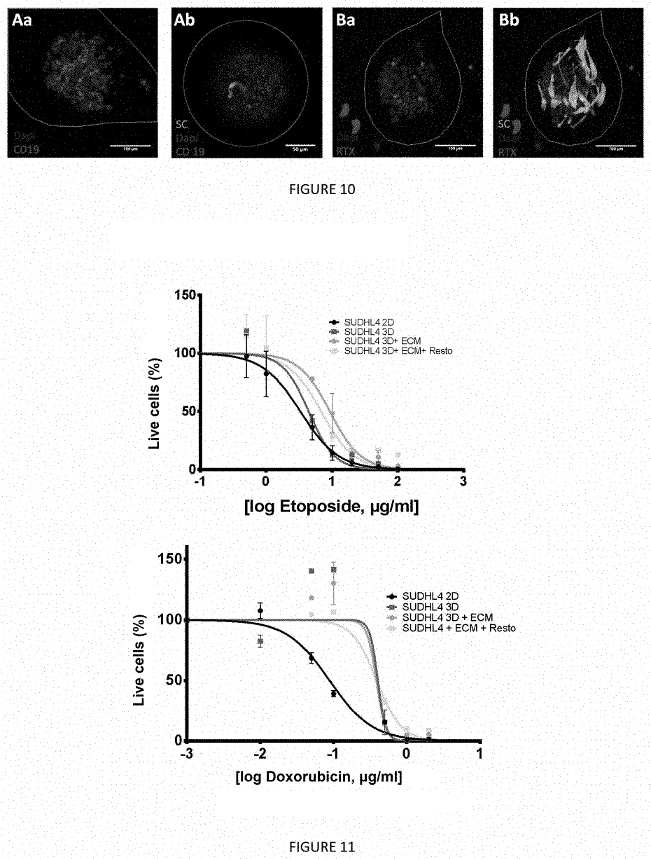

After one night of incubation with ABs, a staining of cells is observed within the spheroids (FIG. 10). This shows that the 3D model according to the invention is suitable for screening therapeutic ABs.

C] Response to Drugs

As shown above, drug diffusion is altered in the 3D structures according to the invention. The purpose of the present experiment is to verify whether this alteration is correlated with chemoresistance. For this purpose, the efficacy of two conventional chemotherapies (doxorubicin and etoposide) was tested in parallel on cells grown in suspension (2D) and on cell clusters from cellular microcompartments according to the invention, containing lymphomatous cells alone or in the presence of ECM, with or without Resto stromal cells, treated with increasing concentrations of these chemotherapy molecules.

After 48 h of incubation, cell survival was assessed by measuring intracellular ATP using the CellTiter-Glo® 3D Cell Viability Assay Kit (Promega).

The results show that the cells organized in cell clusters (3D), which have a structure closer to the structure of a tumour (lymphomatous cells+ECM ± Resto), are less sensitive to conventional chemotherapies than the same cells grown in suspension (FIG. 11 and Table 1).

This confirms that the 3D model according to the invention is more relevant for screening anticancer drugs than suspended cells.

TABLE 1
IC50 of the different drugs by study design used

| | 2D | 3D | 3D + ECM | 3D + ECM + Resto |
|---|---|---|---|---|
| Doxorubicin IC50 μg/ml | 0.09 | 0.4 | 0.4 | 0.4 |
| Etoposide IC50 μg/ml | 3.2 | 4.4 | 9.3 | 7 |

1-20. (canceled)

21. A process for preparing a cellular microcompartment comprising an aggregate of cells containing malignant haematopoietic cells encapsulated in a hydrogel layer, wherein a hydrogel solution and a cell solution comprising malignant haematopoietic cells are concentrically coextruded and then crosslinked.

22. The process for preparing a cellular microcompartment according to claim 21, wherein the malignant haematopoietic cells are lymphomatous cells.

23. The process for preparing a cellular microcompartment according to claim 22, wherein the cell solution further comprises lymphoid stromal cells and extracellular matrix.

24. The process for preparing a cellular microcompartment according to claim 21, wherein the malignant haematopoietic cells are leukaemic cells.

25. The process for preparing a cellular microcompartment according to claim 24, wherein the cell solution further comprises medullary stromal cells and extracellular matrix.

26. The process for preparing a cellular microcompartment according to claim 21, wherein the cell density in the cell solution is between 10·10⁶ and 100·10⁶ cells/mL.

27. The process for preparing a cellular microcompartment according to claim 21, wherein the number ratio of malignant haematopoietic cells to stromal cells in the cell solution is between 1:1 and 1:2.

28. The process for preparing a cellular microcompartment according to claim 23, wherein the cell solution comprises between 1 and 90 vol % cells and between 10 and 99 vol % extracellular matrix.

29. The process for preparing a cellular microcompartment according to claim 25, wherein the cell solution comprises between 1 and 90 vol % cells and between 10 and 99 vol % extracellular matrix.

30. The process for preparing a cellular microcompartment according to claim 21, said process comprising one additional step selected from the group consisting of:
applying a potential of +2 kV to the hydrogel solution; and
generating an electric field between coextrusion means and the crosslinking solution.

31. The process for preparing a cellular microcompartment according to claim 21, said process comprising the additional steps consisting of:
applying a potential of +2 kV to the hydrogel solution; and
generating an electric field between coextrusion means and the crosslinking solution.

32. The process for preparing a cellular microcompartment according claim 21, said process comprising one subsequent step selected from the group consisting of:
freezing the cellular microcompartments; and
hydrolysing the hydrogel capsule of the cellular microcompartments to recover the cell aggregate.

33. The process for preparing a cellular microcompartment according claim 21, said process comprising the subsequent steps consisting of:
freezing the cellular microcompartments; and
hydrolysing the hydrogel capsule of the cellular microcompartments to recover the cell aggregate.

34. A cellular microcompartment obtainable by the process according to claim 21, wherein said microcompartment forms a closed three-dimensional structure comprising an aggregate of cells forming a cohesive cluster constrained in the internal volume of the microcompartment, said aggregate comprising at least malignant haematopoietic cells, encapsulated in an outer hydrogel layer.

35. The cellular microcompartment according to claim 34, wherein said microcompartment consists of an aggregate of lymphomatous cells encapsulated in a hydrogel layer, or an aggregate of leukaemic cells encapsulated in a hydrogel layer.

36. The cellular microcompartment according to claim 34, wherein said microcompartment further comprises an extracellular matrix layer between the cell aggregate and the hydrogel layer and wherein the cell aggregate further comprises stromal cells.

37. The cellular microcompartment according to claim 36, wherein the number ratio of malignant haematopoietic cells to stromal cells in the cell aggregate is between 1:1 and 1000:1.

38. The cellular microcompartment according to claim 34, wherein the outer layer comprises alginate.

39. The cellular microcompartment according to claim 35, wherein the lymphomatous cells are selected from the group consisting of cell lines or purified tumour cells of follicular lymphoma, diffuse large B-cell lymphomas, Burkitt lymphoma, mantle cell lymphoma, peripheral T-cell lymphoma, lymphoblastic lymphoma, anaplastic lymphoma, marginal zone lymphoma, lymphoma of mucosa-associated lymphoid tissue (MALT), lymphoplasmacytic lymphoma, lymphoma of the spleen, cutaneous B-cell lymphomas, cutaneous T-cell lymphomas.

40. The cellular microcompartment according to claim 34, wherein said microcompartment has a diameter or a smallest dimension between 50 μM and 1000 μm.

41. The cellular microcompartment according to claim 34, wherein the cell density is between one hundred and several thousand cells per microcompartment.

42. A cellular microcompartment comprising an aggregate of cells encapsulated in a hydrogel layer, wherein the aggregate of cells comprises malignant haematopoietic cells, and stromal cells, said microcompartment further comprising an extracellular matrix layer between the aggregate of cells and the hydrogel layer.

43. A method for screening or identifying a compound for the treatment of lymphoma comprising the steps:
(a′) bringing cellular microcompartments according to claim 34 into contact with a compound to be tested;
(b) selecting a compound among the compounds capable of at least partially inhibiting the growth of the cell aggregate of said cellular microcompartment and the compounds capable of at least partially killing the cells of the cell aggregate of said cellular microcompartment.

44. The method for screening or identifying a compound for the treatment of lymphoma according to claim 43, said method comprising the preliminary step of:
(a) dissolving the hydrogel layer of the cellular microcompartments before step (a′).
